(19) United States
(12) Patent Application Publication
(10) Pub. No.: US 2005/0059898 A1
(43) Pub. Date: Mar. 17, 2005

(54) Device for determining sleep profiles

(76) Inventor: Rene Gobel, Walldorf (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/496,175

(22) PCT Filed: Nov. 13, 2002
(86) PCT No.: PCT/DE02/04199

(30) Foreign Application Priority Data
Nov. 20, 2001 (DE) 101 56 556.9

(51) Int. Cl. A61B 5/04

(52) U.S. Cl. 600/544

(57) ABSTRACT

A device for the determination of sleep profiles is described. The device has an electrode unit, a signal recording unit and a signal processing/signal evaluating unit separated from the signal recording unit. The signals obtained from the electrode unit are stored on a storage unit adapted to be taken out from and to be replaced in the signal recording unit. The storage unit is supplied to the separated signal processing/signal evaluating unit and is read into the same there. If the signal processing/signal evaluating modalities are modified with this device no modification of the hardware is necessary but only the software of the signal processing/signal evaluating unit has to be modified.

The present invention is directed to a device for the determination of sleep profiles.

Such a device is known from WO 98/22019. The known device has an electrode unit having the form of an electrode band with pre-amplifier operating on the basis of a single frontal EEG channel (active electrode) and placed in the front range symmetrically with respect to the root of the nose. The electrode unit is connected to an autonomously operating microprocessor-controlled signal receiving and analyzing unit. In this unit the EEG signal is compressed and stored in a feature-related manner and after this pre-processing transferred to a computer wherein the classification according to the sleeping phases is carried out in the computer through a population of neuronal networks.

Accordingly, with the known device a pre-processing of the obtained data is carried out in the signal receiving and analyzing unit before the data are transferred to the real signal processing/signal evaluating unit in the form of a PC. This constellation has the disadvantage that, if a modification of the signal processing/evaluating modalities is desired, an extensive modification of the hardware in the device is necessary, namely a modification or replacement of the signal receiving and analyzing unit. Furthermore, with the known device the real raw signal is not maintained but is immediately pre-processed in the signal receiving and analyzing unit so that one cannot refer to the raw signal later on.

It is the object of the invention to provide a device for the determination of sleep profiles which can be used in a versatile manner and with which a modification of the signal processing/evaluating modalities is possible in a simple manner.

According to the invention this object is achieved by a device for the determination of sleep profiles comprising

an electrode unit;

a signal recording unit;

a replaceable storage unit adapted to be taken out from the signal recording unit; and

a signal processing/signal evaluating unit separated from the signal recording unit and into which the storage unit is readable.

Accordingly, with the inventive solution the signal recording unit and the signal processing/signal evaluating unit are disposed separately. Accordingly, if a modification of the signal processing/evaluating modalities is desired only a modification in the signal processing/signal evaluating unit has to be carried out, for instance only a modification of the necessary software, while the signal recording unit is not affected by this, i.e. the raw signal is maintained. This raw signal is recorded on the replaceable storage unit which is taken out from the signal recording unit and from which the recorded raw signal is read into the signal processing/signal evaluating unit.

The inventive device is used not only for sleep laboratories but also for domestic use. The device can be handled in a simple manner. For instance, after the realized signal recording, the storage unit is taken out from the signal recording unit and given to the medical personnel of a sleep laboratory etc. who will then carry out the real signal evaluation process by means of the signal processing/signal evaluating unit and will determine the sleep profiles.

Accordingly, the inventive device enables a direct access to the raw data so that several analysis and classification algorithms of the signal processing/signal evaluating unit can operate with these data. The raw data can be directly screened by the skilled medical personnel either.

The use of new, extended and improved algorithms (evaluation modalities) is possible at any time since for this only the software which is installed on the signal processing/signal evaluating unit, for instance a PC, has to be replaced and no hardware modification is necessary. This new software can be used for already present recordings either since the raw data were stored. In this manner a high extent of flexibility and service-kindness of the device is achieved.

The signal recording unit can be used for the recording of different signals (EEG, ECG, EMG, EOG, oxygen saturation, flow etc.) without making necessary a modification of the hardware. It can be only necessary to use another electrode unit.

Since the data are laid upon a replaceable storage unit (replaceable storage medium) in the signal recording unit, a simple and fast data communication between signal recording unit and signal processing/signal evaluating unit (PC) is made possible. Furthermore, this replaceability enables an enhancement of the data recording time without data transfer to the signal processing/signal evaluating unit by the use of several storage units (storage media). So, it is possible, for instance, to place at the disposal of the patient a plurality of storage units (storage media) so that a data detection can be carried out during several days without making necessary that the patient appears in the hospital or before the doctor. Furthermore, it is possible to exchange only the storage units (storage media) between the doctor or the hospital and the patient whereby

a continuous recording of the data over a very long period of time is possible.

The use of the signal recording unit is very simple and substantially maintenance-free. Only a connection of the unit to the electrode unit is necessary if this has not already been carried out in the rooms of the doctor or in the hospital. Only with recordings over several days a regular change of the current supply (accumulators) and with a correspondingly high sensing rate a change of the storage unit are necessary which are very simple processes.

Accordingly, the inventive solution offers the following advantages:

Flexible, exchangeable evaluating and analyzing algorithms,

problem-free upgrading of the software,

flexible use of the signal recording unit for different signals,

recording and storing of raw data,

fast and simple data transfer between the signal recording unit and the signal processing/signal evaluating unit,

autonomously operating signal recording unit,

ambulant use,

simple use,

large recording capacity,

possibility of recordings over several days.

The real evaluation can be carried out in the signal processing/signal evaluating unit differently, especially in known manner on the basis of neuronal networks or a cluster analysis. In this manner it is pointed to the evaluation method described in the above-cited WO 98/22019 the disclosure of which is herewith incorporated. The inventive device offers the possibility to change from known methods to novel evaluation methods, if such novel evaluation methods have been developed, without having to carry out extensive hardware-like modifications with the device. In these cases substantially only the software designed for the signal processing/signal evaluating unit (PC) has to be modified.

The storage unit (the storage medium) is preferably a multimedia, card. This has the advantage of an especially simple handling. The signal recording unit is formed such that the multimedia card can be plugged into the same in a simple manner for the storage of signals. A transfer of the data stored on the multimedia card to the signal processing/signal evaluating unit is possible by taking out the multimedia card from the signal recording unit and plugging the same into the signal processing/signal evaluating unit.

Generally, the signal processing/signal evaluating unit includes a reading device, an interface or a drive for reading-in the storage unit.

Preferably, the signal recording unit is formed especially simply and has a filter stage, an A/D converter stage, a microcontroller and a current supply. Accordingly, with this unit the signal supplied by the electrode unit is only filtered and A/D converted, i.e. the filtered and A/D converted raw signal is stored in the storage unit. If necessary, an amplifier can be present. Preferably, the current supply is formed by accumulators so that the device can operate independently from the network.

In order to design the signal recording unit as simple as possible the same has preferable at most an event key as access possibility for the user. By this, it is assured that the user cannot manipulate the device and only access possibilities for skilled personnel exist.

As regards the signal processing /signal evaluating unit, this is preferably a conventional PC including the corresponding software for the generation of the sleep profiles. The signal processing evaluation is carried out according to the following scheme, for instance:

Artefact recognition,

extraction of features,

pre-processing,

classification.

According to a special embodiment the inventive solution provides that the signal recording unit is pre-programmable by the signal processing/signal evaluating unit through the storage unit. In this manner, the function of the electrode unit (active electrode) can be controlled (programmed) by skilled personnel. Then the real data detecting/measuring step runs automatically so that the patient has not to interfere with the process. It is important that reprogrammations of corresponding parameters can be realized in this manner. So, for instance, the sensing rate, the start/end time, data of patients, the kind of recording (EEG, ECG etc.), the time, the software of the microcontroller (modification, update etc.) of the signal recording unit can be input or modified.

As regards the electrode unit of the inventive device, any electrode systems can be used. For instance, the electrodes can be arranged on an electrode carrier or so-called direct electrodes can be used. A pre-amplifier cable integrated into the electrode carrier. If direct electrodes are used the pre-amplifier can be integrated into the electrode cable. As minimum the invention provides a one-channel recording with three electrodes and a centrally disposed reference electrode. The central region of the human head is preferred as application range of the electrodes; the front portion is possible either.

A preferred solution includes an electrode band having three up to six electrodes. Press buttons for the contact with the one-way adhesive electrodes and possibly a pre-amplifier are integrated into this front band. Three electrodes are provided in the front range while the additional three electrodes can be provided in the ear range or in the region between ear and eye. The corresponding electrodes can be combined with one another arbitrarily.

Another solution is directed to the provision of pre-switching boxes in connection with three up to eight single electrodes. If necessary, a pre-amplifier, an adapter circuit for a flow sensor or an adapter circuit for a pulse oximeter can be arranged within the pre-switching box. The press buttons for the contact with the one-way adhesive electrodes are located at pre-confectionated cables.

Another possibility consists in the provision of cables which are splitted into individual wires at one side wherein the press buttons for the contact with the one-way adhesive electrodes are located at the end of the individual wires. Possibly, a pre-amplifier-is integrated into the cable.

Electrode positions from the ten-twenty-system can be used either.

Furthermore, a measurement of the EMG (electromyogram) at the legs is possible by means of a bipolar or unipolar derivation. Hereby, the electrodes are adhered to the muscle bulge.

Now, the invention is described in detail by means of an embodiment in connection with the drawing. Of the drawing

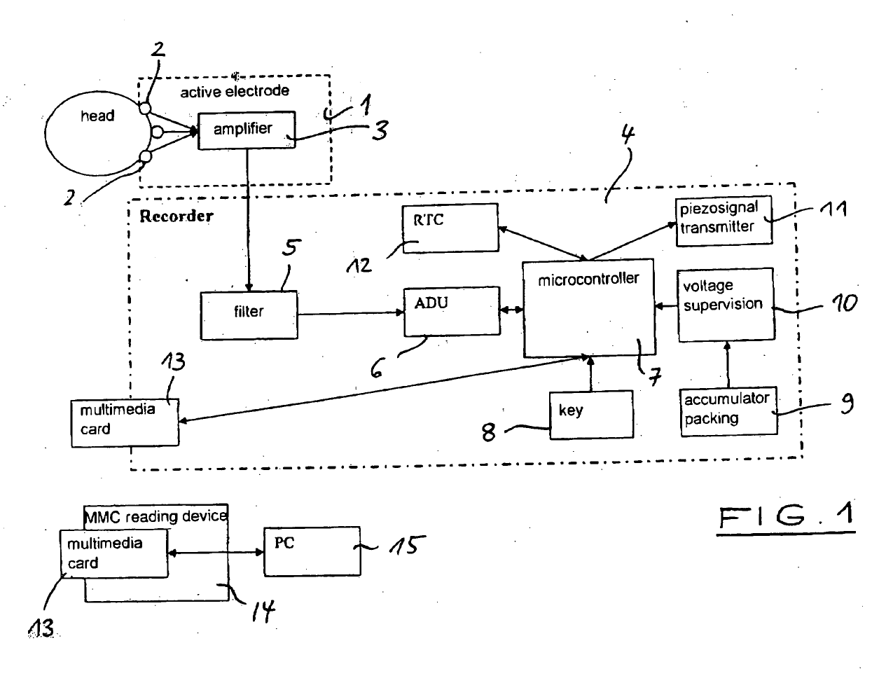

FIG. 1 shows a schematical block representation of the design of a device for the determination of sleep profiles;

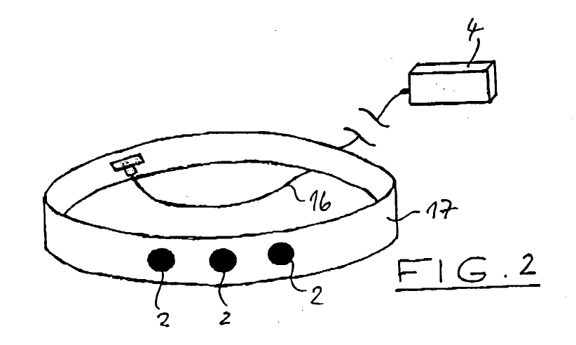

FIG. 2 shows a schematical representation of the electrode arrangement of the device shown in FIG. 1; and

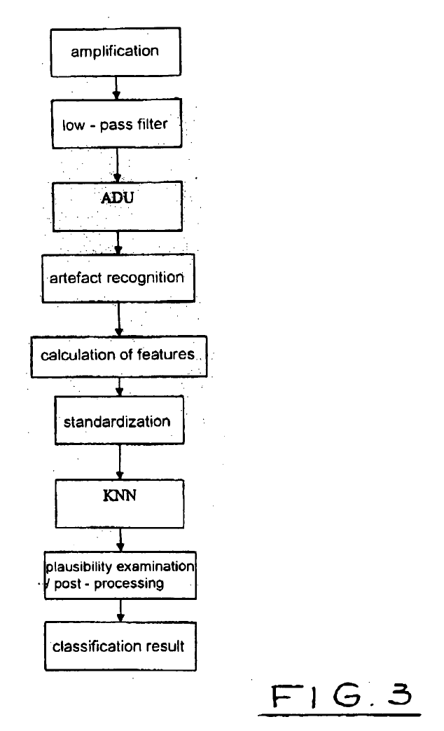

FIG. 3 shows a flow chart of the signal processing/evaluation of the device of FIG. 1.

FIG. 1 shows the schematical design of a device for the determination of sleep profiles in the form of a block diagram. The device is formed by four units, namely an electrode unit 1, a signal recording unit 4, a replaceable storage unit 13 and a signal processing/signal evaluating unit 15. According to this embodiment the replaceable storage unit 13 is formed as multimedia card and the signal processing/signal evaluating unit 15 is formed as personal computer. Furthermore, the device includes a reading device 14 by means of which the stored data on the storage unit 13 can be read into the signal processing/signal evaluating unit 15.

According to this embodiment, the schematically shown electrode unit (active electrode) 1 has three electrodes 2 which can be applied to the front region of the head of a patient as well as an amplifier 3. The amplifier 3 can be integrated, for instance, into the electrode carrier if such a carrier is present or into the electrode cables. It can be formed as pre-amplifier either.

The amplified signals of the electrode unit 1 are supplied through a suitable electrical cable to the recording unit 4 which has a microcontroller 7 for the control of the respective stages of the recording unit. The amplified signal from the electrode unit 1 is firstly supplied to a filter stage 5 (low-pass filter) and is filtered there. Then it is supplied to an ADU stage 6 (combination consisting of an A/D converter and low-pass filter) and is filtered there again and A/D converted. Thereafter, it is supplied to the microcontroller 7 as digital signal. On the microcontroller 7 it is supplied to the storage unit 13 (multimedia card) and is stored there. The patient has the possibility to control the microcontroller 7 by means of an event key 8. The current supply of the microcontroller 7 is carried out by an accumulator packing 9 with post-arranged voltage supervision 10. Furthermore, the signal recording unit 4 has a piezosignal transmitter 11 and a RTC stage 12 (time) which are associated with the microcontroller 7.

The storage unit 13 (multimedia card) containing the stored signal can be taken out from the signal recording unit 4 and can be plugged into a reading device 14 with which the stored data can be read into the signal processing/signal evaluating unit 15 which is a conventional personal computer. The signal processing and signal evaluation to obtain the desired sleep profiles is carried out with the unit 15.

FIG. 2 shows the schematical design of an electrode unit. This has a front band 17 into which the three electrodes 2 are integrated. An electrical cable 16 leads from the front band 17 to the signal recording unit 4. The amplifier 3 shown in FIG. 1 can be integrated into the front band 17 or into the cable 16.

Finally, FIG. 3 shows a flow chart for the signal processing/signal evaluation. The signal amplified in a first stage and filtered in a second stage is A/D converted in the ADU stage and is stored as digital signal in the storage unit. Then it is transferred to the real signal processing/signal evaluating unit 15 which carries out with the suitable software a calculation of features, a standardization, a KNN processing and a plausibility examination/post-processing. A corresponding classification result is supplied. Of course, the signal processing/signal evaluating unit 15 can be provided with a suitable display unit in order to enable an optical representation of the determined sleep profiles.

1. A device for the determination of sleep profiles comprising
an electrode unit (1);
a signal recording unit (4);
a replaceable storage unit (13) adapted to be taken out from the signal recording unit (4); and
a signal processing/signal evaluating unit (15) separated from the signal recording unit (4) and into which the storage unit (13) can be read.

2. The device according to claim 1, characterized in that the storage unit (13) is a multimedia card.

3. The device according to claim 1 or 2, characterized in that the signal recording unit (4) includes a filter stage (5), an A/D converter stage (6), a microcontroller (7) and a current supply (9).

4. The device according to one of the preceding claims, characterized in that the signal recording unit (4) has an event key (8).

5. The device according to one of the preceding claims, characterized in that the signal processing/signal evaluating unit (15) is a personal computer.

6. The device according to one of the preceding claims, characterized in that the electrode unit (1) has at least three electrodes (2).

7. The device according to one of the preceding claims, characterized in that the electrode unit (1) has an electrode carrier (17).

8. The device according to one of the preceding claims, characterized in that a pre-amplifier (3) is integrated into the electrode unit (1).

9. The device according to one of the preceding claims, characterized in that the signal processing/signal evaluating unit (15) includes a reading unit (14), an interface or a drive for reading into the storage unit (13).

10. The device according to one of the preceding claims, characterized in that the signal recording unit (4) is controllable or pre-programmable by the signal processing/signal evaluating unit (15) through the storage unit (13).
